(19) United States
(12) Patent Application Publication
(10) Pub. No.: US 2024/0170134 A1
(43) Pub. Date: May 23, 2024

(54) PATIENT ROOM STATUS SEQUENCING MANAGEMENT SYSTEM

(71) Applicant: Igor Mekhtiev, Horace, ND (US)

(72) Inventor: Igor Mekhtiev, Horace, ND (US)

(21) Appl. No.: 18/057,210

(22) Filed: Nov. 19, 2022

Related U.S. Application Data
(60) Provisional application No. 63/384,425, filed on Nov. 19, 2022.

(51) Int. Cl. G16H 40/20; G06F 3/04817; G06F 3/0482

(52) U.S. Cl. CPC G16H 40/20; G06F 3/04817; G06F 3/0482

(57) ABSTRACT

A Patient and Room Status Sequencing Management system includes a server operable to dynamically sequence actions associated with a room status treatment sequence for a patient, a first inner room graphical user interface in communication with the server, a first outer room graphical user interface in communication with the server, the first outer room graphical user interface and the first inner room graphical user interface associated with a single treatment room, the first inner room graphical user interface operable to display actions associated with the treatment that are at a first confidentiality level and the first outer room graphical user interface operable to display actions associated with the treatment that are at a second confidentiality level, the second confidentiality level lesser than the first confidentiality level, and a dashboard graphical user interface in communication with the server and remote from the treatment room, the dashboard graphical user interface operable to display data associated with at least the first outer room graphical user interface.

### BACKGROUND

The present disclosure relates to patient flow management and more specifically to a system and method that optimizes the sequencing of patients though treatment rooms.

Resource and patient flow management may be an important concern. In order to provide effective and efficient management of healthcare environments, healthcare institutions are using a variety of healthcare management systems. In some cases, a balance between the types of treatments that may need to be performed and patient demand may need to be maintained in an efficient manner.

### SUMMARY

A Patient and Room Status Sequencing Management system according to one disclosed non-limiting embodiment of the present disclosure includes a server operable to dynamically sequence actions associated with a room status treatment sequence for a patient; a first inner room graphical user interface in communication with the server; a first outer room graphical user interface in communication with the server, the first outer room graphical user interface and the first inner room graphical user interface associated with a single treatment room, the first inner room graphical user interface operable to display actions associated with the treatment that are at a first confidentiality level and the first outer room graphical user interface operable to display actions associated with the treatment that are at a second confidentiality level, the second confidentiality level lesser than the first confidentiality level; and a dashboard graphical user interface in communication with the server and remote from the treatment room, the dashboard graphical user interface operable to display data associated with at least the first outer room graphical user interface.

A further embodiment of any of the foregoing embodiments of the present disclosure includes that the status of the room on the first outer room graphical user interface comprises changing a color of an entire screen of the first outer room graphical user interface.

A further embodiment of any of the foregoing embodiments of the present disclosure includes that the first inner room graphical user interface is operable to display an action associated with a room status treatment sequence for a patient.

A further embodiment of any of the foregoing embodiments of the present disclosure includes that the action is one of a sequence of actions, the action associated with the status of the room.

A further embodiment of any of the foregoing embodiments of the present disclosure includes that the action comprises a multiple of choices depicted by a multiple of buttons.

A further embodiment of any of the foregoing embodiments of the present disclosure includes that each of the multiple of buttons comprises an icon and text.

A further embodiment of any of the foregoing embodiments of the present disclosure includes that each of the multiple of choices are the only choices available for that action in the sequence of actions.

A further embodiment of any of the foregoing embodiments of the present disclosure includes that at least one of the multiple of choices comprise a timer function that displays a time remaining on the first outer room graphical user interface and the first inner room graphical user interface.

A further embodiment of any of the foregoing embodiments of the present disclosure includes that the room status treatment sequence for the patient is one of a multitude of room status treatment sequences.

A computer-storage media having computer-executable instructions embodied thereon that, when executed, perform a method to dynamically sequence actions associated with a room status treatment sequence for a patient according to one disclosed non-limiting embodiment of the present disclosure includes: receiving a real-time status of a room in response to an input to a first inner room graphical user interface within the room; displaying the status of the room on a dashboard displaced from the room; and identifying the status of the room wherein the status indicates at least whether an action associated with the treatment is completed in response to an input to the first inner room graphical user interface within the room.

A further embodiment of any of the foregoing embodiments of the present disclosure includes displaying the status of the room on a first outer room graphical user interface adjacent to the room.

A further embodiment of any of the foregoing embodiments of the present disclosure includes that displaying the status of the room on the first outer room graphical user interface comprises changing a color of an entire screen of the first outer room graphical user interface.

A further embodiment of any of the foregoing embodiments of the present disclosure includes that the first inner room graphical user interface is operable to display actions associated with the treatment that are at a first confidentiality level and the first outer room graphical user interface operable to display actions associated with the treatment that are at a second confidentiality level, the second confidentiality level lesser than the first confidentiality level.

A further embodiment of any of the foregoing embodiments of the present disclosure includes displaying the action of a sequence of actions associated with a room status treatment sequence for a patient on the first inner room graphical user interface, the action associated with the status of the room.

A further embodiment of any of the foregoing embodiments of the present disclosure includes wherein displaying the action comprises displaying a multiple of choices depicted by a multiple of buttons on the first inner room graphical user interface.

A further embodiment of any of the foregoing embodiments of the present disclosure includes that displaying the multiple of choices as a multiple of buttons on the first inner room graphical user interface, wherein each of the multiple of buttons are labeled with an icon and text.

A further embodiment of any of the foregoing embodiments of the present disclosure includes that each of the multiple of choices are the only choices available for that action in the sequence of actions.

A further embodiment of any of the foregoing embodiments of the present disclosure includes that at least one of the multiple of choices comprise a timer function displaying a time remaining on the first outer room graphical user interface and the first inner room graphical user interface.

The foregoing features and elements may be combined in various combinations without exclusivity, unless expressly indicated otherwise. These features and elements as well as the operation thereof will become more apparent in light of the following description and the accompanying drawings. It should be appreciated however that the following description and drawings are intended to be exemplary in nature and non-limiting.

### BRIEF DESCRIPTION OF THE DRAWINGS

Various features will become apparent to those skilled in the art from the following detailed description of the disclosed non-limiting embodiment. The drawings that accompany the detailed description can be briefly described as follows:

FIG. 1 is a schematic block diagram view of a Patient and Room Status Sequencing Management system according to one disclosed non-limiting embodiment.

FIG. 2 is a block diagram of a method of operation for the Patient and Room Status Sequencing Management system according to one disclosed non-limiting embodiment.

FIG. 3 is a screen shot of a dashboard graphical user interface of the Patient and Room Status Sequencing Management system according to one disclosed non-limiting embodiment.

FIG. 4 is a block diagram of an exam room flow of the Patient and Room Status Sequencing Management system according to one disclosed non-limiting embodiment.

FIG. 5 is a screen shot of a graphical user interface of the Patient and Room Status Sequencing Management system according to one disclosed non-limiting embodiment.

FIG. 6 is a screen shot of a graphical user interface of the Patient and Room Status Sequencing Management system according to one disclosed non-limiting embodiment.

FIG. 7 is a screen shot of a graphical user interface of the Patient and Room Status Sequencing Management system according to one disclosed non-limiting embodiment.

FIG. 8 is a screen shot of a graphical user interface of the Patient and Room Status Sequencing Management system according to one disclosed non-limiting embodiment.

FIG. 9 is a screen shot of a graphical user interface of the Patient and Room Status Sequencing Management system according to one disclosed non-limiting embodiment.

FIG. 10 is a screen shot of a graphical user interface of the Patient and Room Status Sequencing Management system according to one disclosed non-limiting embodiment.

### DETAILED DESCRIPTION

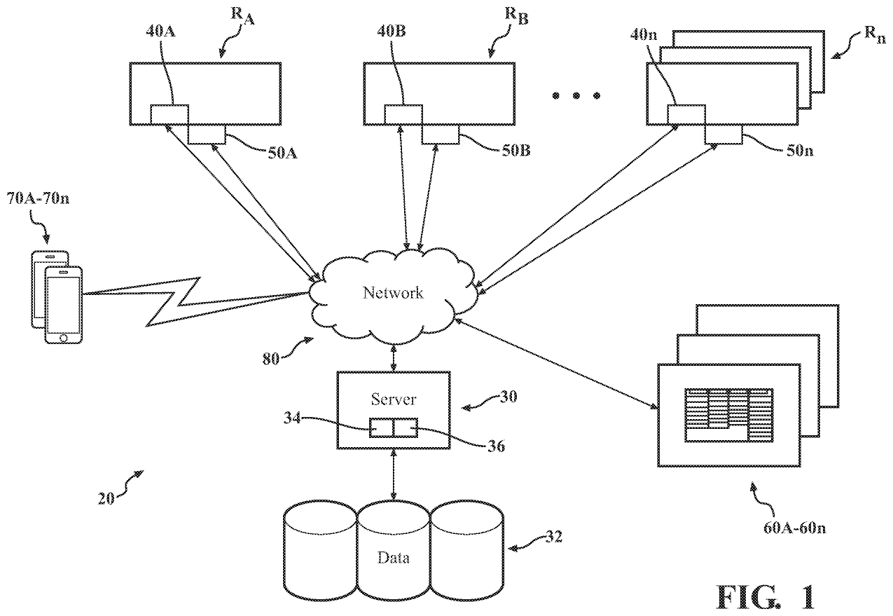

FIG. 1 schematically illustrates a Patient and Room Status Sequencing Management system 20 that provides for an efficient use of an office with multiple treatment rooms R. It should be appreciated that the illustrated system 20 is merely an example of one such environment and is not intended to suggest any limitation as to the scope of use or functionality thereof. Neither should the system 20 be interpreted as having any dependency or requirement relating to any single component or combination of components illustrated therein.

Embodiments of the system 20 may be operational with numerous computing system environments or configurations. Examples of such computing systems, environments, and/or configurations that may be suitable for use with embodiments herein include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

Embodiments of the system 20 may be described in the general context of computer-executable instructions, such as program modules, being executed by a computer. Generally, program modules include, but are not limited to, routines, programs, objects, components, and data structures that perform particular tasks or implement particular abstract data types. The system 20 may also be practiced in distributed computing environments where tasks are performed by remote processing devices that are linked through a communications network. In a distributed computing environment, program modules may be located in local and/or remote computer storage media including, by way of example only, memory storage devices.

The system 20 may be controlled and operated by a single owner. The system generally includes a server 30 that communicates with a multiple of inner room graphical user interfaces 40A-40n, a multiple of outer room graphical user interfaces 50A-50n, one or more dashboard graphical user interfaces 60A-60n, and a multiple of handheld devices 70A-70n over a network 80.

The server 30 typically includes, or has access to, a variety of computer readable media, for instance, database 32. Computer readable media can be any available media that may be accessed by server 30, and includes volatile and nonvolatile media, as well as removable and non-removable media. By way of example, and not limitation, computer readable media may include computer storage media and communication media. Computer storage media may include, without limitation, volatile and nonvolatile media, as well as removable and nonremovable media implemented in any method or technology for storage of information, such as computer readable instructions, data structures, program modules, or other data. For example, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the server 30. The computer storage media, including database 32, may provide storage of computer readable instructions, data structures, program modules, and other data for the server 30.

The server 30 may operate within the computer network 80 using logical connections to the inner room graphical user interfaces 40A-40n, the multiple of outer room graphical user interfaces 50A-50n, one or more dashboard graphical user interfaces 60A-60n, and the multiple of handheld devices 70A-70n. The “graphical user interface” may be located at a variety of locations in a medical, research, or other such environment that includes a multiple of rooms R.

A “graphical user interface” conveys its customary meaning and may include various devices such as screens, tablets, mobile devices, portable electronic devices, handheld devices, personal computers, monitors, etc. A “server” conveys its customary meaning that provides service and/or data connection, e.g., to handheld devices and tablets. The handheld devices and tablets refer to a type of portable electronic device that is at least configured to communicate with the server over a long-range wireless communication network, such as a SMS, wireless, or cellular network, short-range wireless communication network such as Bluetooth, as well as direct hard-wired connections. Examples of handheld devices may include, but are not limited to, mobile phones, portable computers, tablets, and the like.

The network 80 may include, without limitation, local area networks (LANs) and/or wide area networks (WANs). Such networking environments are commonplace in offices, enterprise-wide computer networks, intranets, and the Internet. When utilized in a WAN networking environment, the server 30 may include a modem or other device for establishing communications over the WAN, such as the Internet. In a networked environment, program modules or portions thereof may be stored in the server 30 and/or on any of the graphical user interfaces. For example, and not by way of limitation, various application programs may reside on the memory associated with any one or more of the graphical user interfaces. It will be appreciated that the network connections shown are exemplary and other means of establishing a communications link between the server 30 and the multitude of graphical user interfaces may be utilized.

In operation, a user may enter commands and information into the server 30 or convey the commands and information to the server 30 via one or more of the graphical user interfaces over the network 80. Commands and information may also be sent directly from the server 30. In addition to a monitor, the server 30 and/or graphical user interfaces and/or handheld devices, etc., may include other peripheral input and output devices, such as speakers and a printer.

The multiple of inner room graphical user interfaces 40A-40n and the multiple of outer room graphical user interfaces 50A-50n may alternatively or additionally be in communication with the server 30 via a wired connection with a Power over Ethernet (PoE) injector to form the network 80. The Power over Ethernet (PoE) injector connects PoE-enabled network devices to a non-PoE LAN switch port. More specifically, a PoE injector can be used to connect a wireless access point, IP phone, network camera, graphical user interface, or any IEEE 802.3 af/at-powered device (PD) to a network. The multiple of inner room graphical user interfaces 40A-40n and the multiple of outer room graphical user interfaces 50A-50n may alternatively or additionally be in communication with the server 30 wirelessly, however the power may be provided as such: POE INJECTOR→ETH CABLE→48V/58V buck converter→5V→tablet via USB interface. Alternatively, the communication path may be POE SWITCH→ETH CABLE POWER/DATA→TABLET (no wifi).

Components of the server 30 may include, without limitation, a processing unit, internal system memory, and a suitable system bus for coupling various system components, including the database 32. The system bus may be any of several types of bus structures, including a memory bus or memory controller, a peripheral bus, and a local bus, using any of a variety of bus architectures. Although various internal components of the server 30 and the other devices are not shown, it should be appreciated that such components and their interconnection are known. Accordingly, additional details concerning the internal construction of the server 30 and the associated devices in communication therewith and operation thereof are not further disclosed herein.

The server 30 hosts, for example, at least a software application 34 that includes an application program interface (API) 36 to operate the Patient and Room Status Sequencing Management system 20. An application program interface (API) is a set of routines, protocols, and tools for building software applications that may include a computer-storage media having computer-executable instructions embodied thereon that, when executed, perform a method to dynamically sequence actions associated with a room status treatment sequence for a patient. The API specifies how software elements interact. APIs may be used when programming the graphical user interface components. A server-side web API is a programmatic interface for one or more publicly exposed endpoints to a defined request-response message system

Each treatment room R includes at least one inner room graphical user interface 40A-40n, and one outer room graphical user interface 50A-50n. The inner room graphical user interface 40A-40n is located within its respective treatment room, as such inner room graphical user interfaces 40A-40n may display more confidential information while the respective outer room graphical user interfaces 50A-50n may display information that is less confidential than the inner room graphical user interface 40A-40n as such information may be viewed by others such as other patients who are walking past the room.

Although a particular treatment type may be described as an example herein, it should be appreciated that any type of office with multiple rooms within which various treatments are performed will benefit herefrom to include, but not be limited to, physicians, dentists, optometrists, etc.

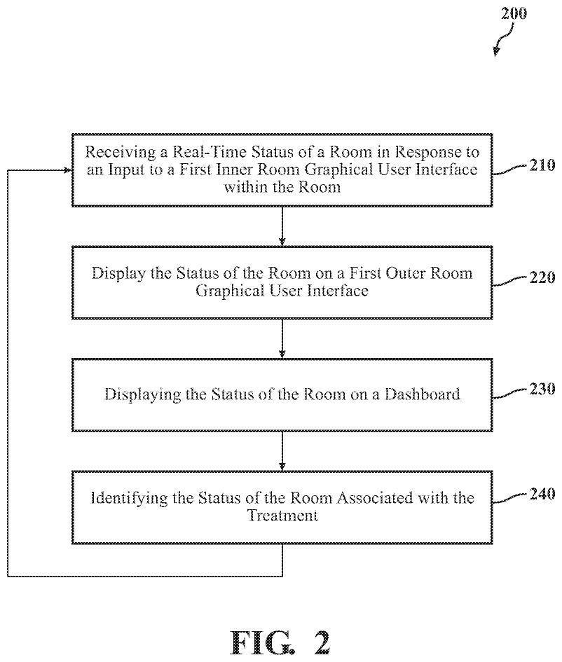

With reference to FIG. 2, a method 200 for operation of the system 20 is disclosed in terms of functional block diagrams. The functions are programmed software routines capable of execution in various microprocessor-based electronics control embodiments and represented herein as example block diagrams. The method dynamically sequences actions associated with a room status treatment sequence within an environment that includes a multiple of treatment rooms.

Initially, the server 30 communicates with the multiple of inner room graphical user interfaces 40A-40n, the multiple of outer room graphical user interfaces 50A-50n, the dashboard graphical user interfaces 60A-60n, and the multiple of handheld devices 70A-70n as input and output devices associated with the associated room R

The application program interface (API) 36 may utilize communications with the multiple of inner room graphical user interfaces 40A-40n and the multiple of outer room graphical user interfaces 50A-50n to track in real time the room status and/or associated patient for each associated room. The tracking provides for an essentially unlimited customizable room status sequencing such that the total number of rooms are utilized to their most efficient capacity. That is, by tracking the rooms based on the treatments and or status thereof, the rooms, and thus the overall office space, are most efficiently utilized.

The method 200 for operation of the system 20 generally includes receiving a real-time status of a room in response to an input to the inner room graphical user interface 40A within the room R (210). The inner room graphical user interface 40A is operable to display each action of a sequence of actions associated with a room status treatment sequence action that is associated with the status of the room. The action may include displaying a multiple of choices depicted by a multiple of buttons on the first inner room graphical user interface 40A.

The multiple of choices may be displayed as a multiple of buttons on the inner room graphical user interface 40A wherein each of the multiple of buttons are labeled with an icon and text. Each of the multiple of choices are displayed as the only choices available for that action in the sequence of actions. That is, since each consecutive screen is essentially self-explanatory, the choices presented to the user are the only choices available to them during that action such that there is no confusion associated with a room status treatment sequence for that patient. The user never then has to consider how to identify the status of the room as such status is integrated into the selections provided. For example, at least one of the multiple of choices may be a timer function displaying a time remaining on the outer room graphical user interface 50A and the inner room graphical user interface 40A. Selection of that choice thereby not only starts the timer function but also updates the status, color of the screen, etc., which are reflected across every device and dashboard of the system 20.

Next, the status of the room is displayed (220) on the outer room graphical user interface 50A adjacent to that room R. Displaying the status of the room on the outer room graphical user interface 50A may include changing a color of an entire screen so that it is readily identified from a distance.

The status of the room R may also be concurrently displayed (230; FIG. 3) on one or more of the dashboard graphical user interfaces 60A-60n displaced from the room R. The dashboard graphical user interface 60A may be located in a central position such as a nurse's station, etc. The dashboard graphical user interfaces 60A-60n may change layouts dynamically depending on screen size and may include a dynamic floorplan map which will reflect the colors and room statuses.

The status of the room R is thereby readily identified (240) for the room status treatment sequence for that patient. The status indicates at least whether an action associated with the treatment is completed in response to an input to the inner room graphical user interface 40A. For example, once the timer function is completed, the status of the room automatically changes and is thereby identified so that the next multiple of choices are displayed as the only choices available for that next action in the sequence of actions.

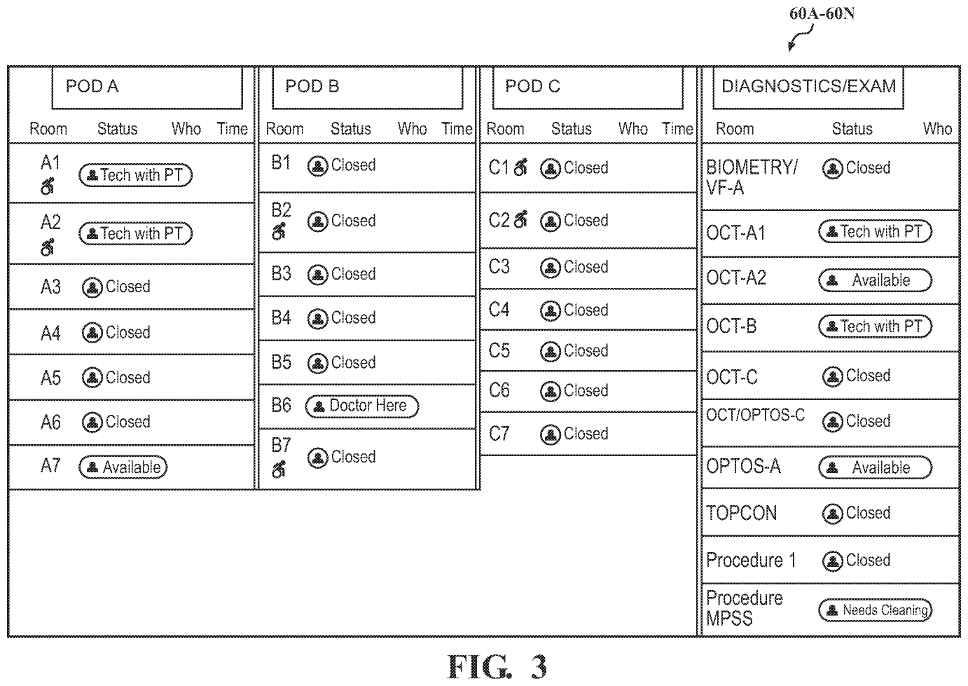

In an example, the customizable room status sequencing may include an exam room flow 300 (FIG. 4) with dynamically sequenced actions associated with a room status treatment sequence for a patient. The customizable room sequencing system 20 is essentially unlimited in that it is predicated on the system's ability to optimize clinical office practices by establishing multiple predefined room status process flows whereby each process flow serves as a unique tree of selections with customizable room sequencing tree branches, in which each tree branch dynamically sequences specific actions and related room statuses. Although the illustrated exam room flow 300 are disclosed as particular examples, it should be appreciated that alternative and/or additional flows may be provided to facilitate efficient sequencing. Furthermore, various other paths other than the example path related to a dilation are provided.

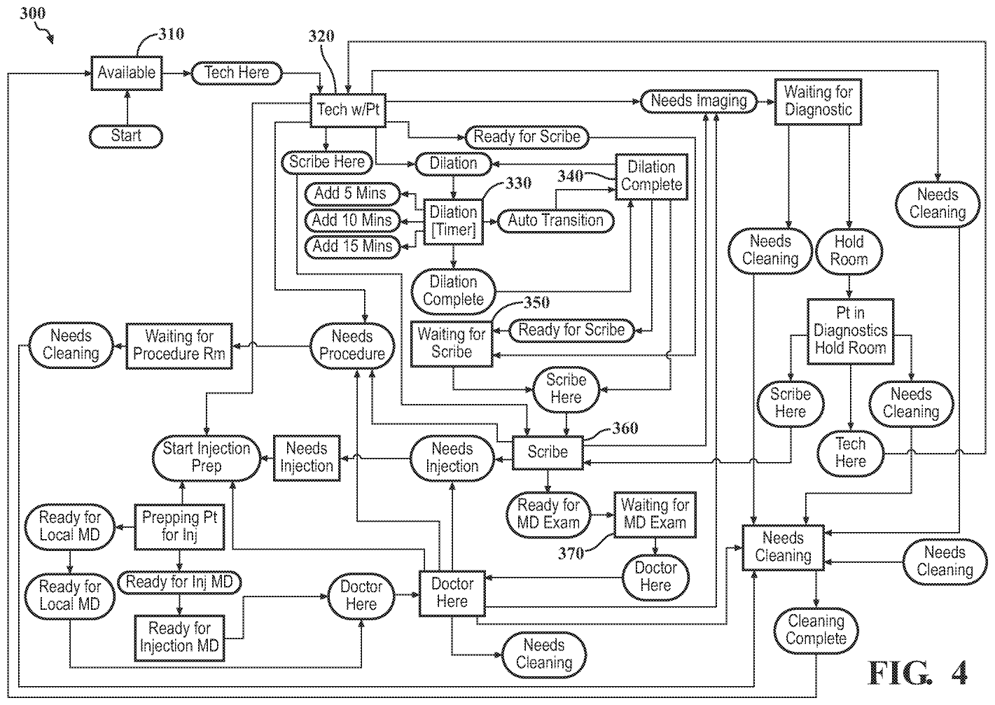

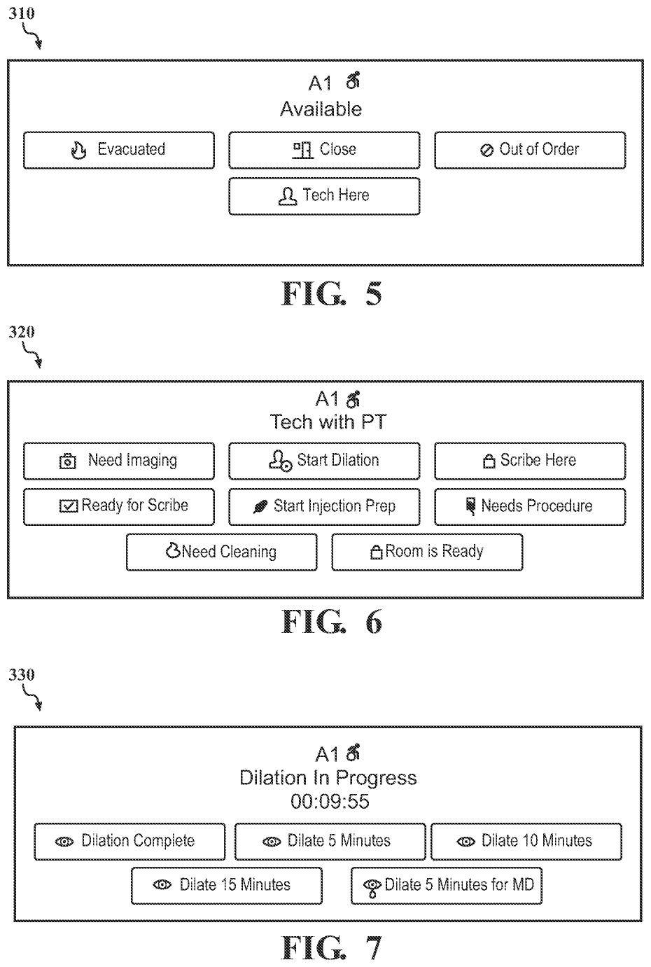

With reference to FIG. 4, in the exam room flow 300 example, an AVAILABLE screen 310 (FIG. 5) may initially be displayed on the inner and outer room graphical user interfaces 40A-40n, 50A-50n of a room R, referred to as A1. The outer room graphical user interfaces 50A-50n may include a color changing status alert mechanism that change based on room or patient status. For example, the AVAILABLE screen 310 on the outer room graphical user interface 50A may be, for example, entirely black. The color coding of the screen permits one who is, for example, looking down a hallway of rooms to immediately be able to identify what rooms are available. That is, the screen color operates as a readily visible indicator of status.

The AVAILABLE screen 310 (FIG. 5) provides selections that are associated with the room flow. That is, only selections in accord with prior selections are displayed. This provides a guided flow based on the room status/room type and patient type so as to essentially alleviate any training required for performance of the flow. The selections in this example may include: TECH HERE; CLOSE; and OUT OF ORDER which are represented as buttons with an associated icon and a label. In this example, when the tech arrives the TECH HERE selection is made. Although particular selections are displayed in this example, it should be appreciated that additional or other selections may be provided which will result in other paths in the exam room flow 300 based on that selection.

The exam room flow 300 then moves to the TECH WITH PT (tech with patient) screen 320 (FIG. 6). The TECH WITH PT screen 320 then provides selections which may include: NEEDS IMAGING; START DILATION; SCRIBE HERE; READY FOR SCRIBE; START INJECTION PREP; NEEDS PROCEDURE; NEEDS CLEANING; and ROOM IS READY. As mentioned above, the user is only presented with selections available to them at any given step as designed by the clinical staff/practice to accommodate their needs for the room status treatment sequence. If the tech is with the patient, the tech is provided with selections that they can perform based on their duties. This promotes the lean flow of the practice and reduces decision fatigue.

In this example, the tech may select START DILATION such that the exam room flow 300 moves to the DILATION IN PROGRESS screen 330 (FIG. 7). The DILATION IN PROGRESS screen 330 may provide multiple dilation time selections as well as an associated countdown timer. This automatically starts the timer which can be modified if needed. In this example, the outer room graphical user interface 50A may also include the color changing status alert mechanism and display the countdown timer. For example, when the countdown timer is running the screen may be yellow, but when complete or within a certain time period prior to completion change to green so it is readily apparent from the outside of the room the status of the dilation. At this point, the staff may also be alerted by the dashboard that the PT is ready to be seen.

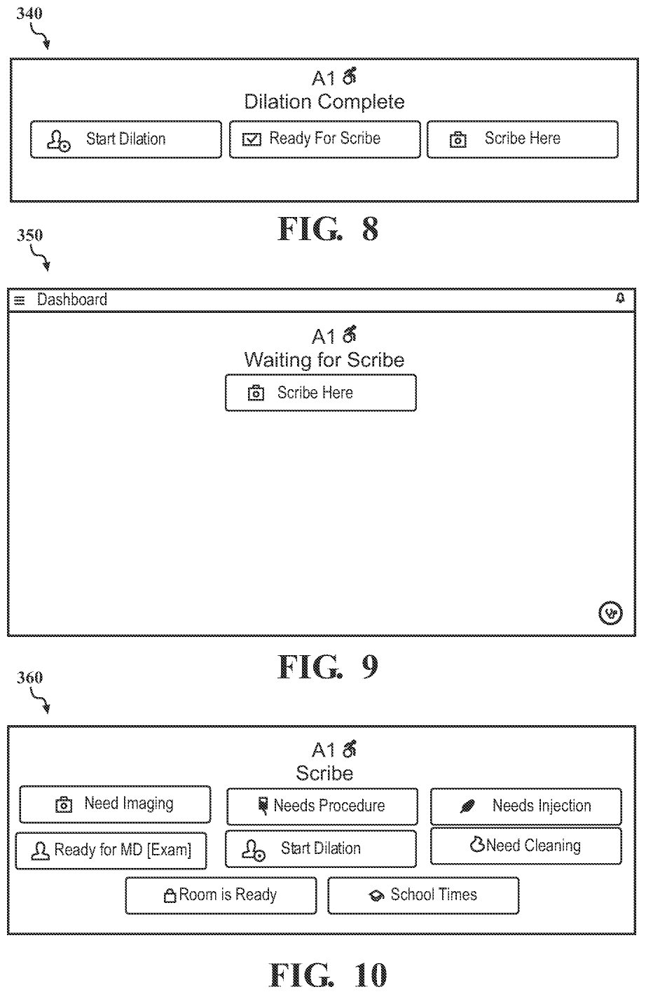

In this example, once the dilation is complete, the exam room flow 300 moves to the DILATION COMPLETE screen 340 (FIG. 8). The DILATION COMPLETE screen 340 then provides selections which may include: START DILATION; READY FOR SCRIBE; and SCRIBE HERE.

The next selection may be READY FOR SCRIBE such that the exam room flow 300 moves to the WAITING FOR SCRIBE screen 350 (FIG. 9) then to the SCRIBE screen 360 (FIG. 10). The WAITING FOR SCRIBE screen 350 (FIG. 9) may have a single selection and also be located on the outer room graphical user interfaces 50A-50n since it has no confidential information and serves as an alert.

The SCRIBE screen 360 then provides selections which may include: NEED IMAGING; NEEDS PROCEDURE; NEEDS INJECTION; READY FOR MD (EXAM); START DILATION; NEED CLEANING; ROOM IS READY; SCHOOL TIME; etc. After the SCRIBE screen 360 the application progresses to the next predetermined status such as WAITING FOR MD screen 370, or it can be interrupted if needed.

In an alternative embodiment, these screens and views are available on any handheld devices 70A-70n. The doctor can dedicate several defined rooms as the ones being in the surgery center. This way, staff can observe the status of the doctor in the surgery center and prep the patient when the case is over.

In an alternative embodiment, the doctor or other professional can refer to his handheld devices 70A-70n to follow which patients are ready, instead of constantly having to walk to a central status board such as may be at a nurse's station or central office.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason the appended claims should be studied to determine true scope and content.

What is claimed is:

1. A Patient and Room Status Sequencing Management system, comprising:
a server operable to dynamically sequence actions associated with a room status treatment sequence for a patient;
a first inner room graphical user interface in communication with the server;
a first outer room graphical user interface in communication with the server, the first outer room graphical user interface and the first inner room graphical user interface associated with a single treatment room, the first inner room graphical user interface operable to display actions associated with the treatment that are at a first confidentiality level and the first outer room graphical user interface operable to display actions associated with the treatment that are at a second confidentiality level, the second confidentiality level lesser than the first confidentiality level; and
a dashboard graphical user interface in communication with the server and remote from the treatment room, the dashboard graphical user interface operable to display data associated with at least the first outer room graphical user interface.

2. The system as recited in claim 1, wherein the status of the room on the first outer room graphical user interface comprises changing a color of an entire screen of the first outer room graphical user interface.

3. The system as recited in claim 1, wherein the first inner room graphical user interface is operable to display an action associated with a room status treatment sequence for a patient.

4. The system as recited in claim 3, wherein the action is one of a sequence of actions, the action associated with the status of the room.

5. The system as recited in claim 4, wherein the action comprises a multiple of choices depicted by a multiple of buttons.

6. The system as recited in claim 5, wherein each of the multiple of buttons comprises an icon and text.

7. The system as recited in claim 6, wherein each of the multiple of choices are the only choices available for that action in the sequence of actions.

8. The system as recited in claim 7, wherein at least one of the multiple of choices comprise a timer function that displays a time remaining on the first outer room graphical user interface and the first inner room graphical user interface.

9. The system as recited in claim 3, wherein the room status treatment sequence for the patient is one of a multitude of room status treatment sequences.

10. A computer-storage media having computer-executable instructions embodied thereon that, when executed, perform a method to dynamically sequence actions associated with a room status treatment sequence for a patient, the method comprising:
receiving a real-time status of a room in response to an input to a first inner room graphical user interface within the room;
displaying the status of the room on a dashboard displaced from the room; and
identifying the status of the room wherein the status indicates at least whether an action associated with the treatment is completed in response to an input to the first inner room graphical user interface within the room.

11. The method as recited in claim 10, further comprising displaying the status of the room on a first outer room graphical user interface adjacent to the room.

12. The method as recited in claim 11, wherein displaying the status of the room on the first outer room graphical user interface comprises changing a color of an entire screen of the first outer room graphical user interface.

13. The method as recited in claim 12, wherein the first inner room graphical user interface operable to display actions associated with the treatment that are at a first confidentiality level and the first outer room graphical user interface operable to display actions associated with the treatment that are at a second confidentiality level, the second confidentiality level lesser than the first confidentiality level

14. The method as recited in claim 10, further comprising displaying the action of a sequence of actions associated with a room status treatment sequence for a patient on the first inner room graphical user interface, the action associated with the status of the room.

15. The method as recited in claim 14, wherein displaying the action comprises displaying a multiple of choices depicted by a multiple of buttons on the first inner room graphical user interface.

16. The method as recited in claim 15, wherein displaying the multiple of choices as a multiple of buttons on the first inner room graphical user interface, wherein each of the multiple of buttons are labeled with an icon and text.

17. The method as recited in claim 16, wherein each of the multiple of choices are the only choices available for that action in the sequence of actions.

18. The method as recited in claim 17, wherein at least one of the multiple of choices comprise a timer function displaying a time remaining on the first outer room graphical user interface and the first inner room graphical user interface.
